(19) United States
(12) Patent Application Publication
(10) Pub. No.: US 2005/0165233 A1
(43) Pub. Date: Jul. 28, 2005

(54) Continuous process for producing hydroxyazapirones by oxidation

(76) Inventors: Mourad Hamedi, Eatontown, NJ (US); Thomas L. LaPorte, Kendall Park, NJ (US); Daniel J. Watson, Lafayette, CO (US); Lifen Shen, North Brunswick, NJ (US); Eric D. Dowdy, Foster City, CA (US); Jeffrey S. Depue, Hillsborough, NJ (US); Yeung Yu Chan, Kendall Park, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/988,791

(22) Filed: Nov. 15, 2004

Related U.S. Application Data
(60) Provisional application No. 60/520,844, filed on Nov. 18, 2003.
(60) Provisional application No. 60/541,409, filed on Feb. 3, 2004.

(51) Int. Cl. C07D 43/14

(52) U.S. Cl. 544/295; 544/230

(57) ABSTRACT

A method for continuous production of hydroxyazapirones, for example 6-hydroxybuspirone, using a modified reactor. The products of the invention are useful as pharmaceutical agents.

This applications claims the benefit of priority of U.S. Provisional Application Ser. Nos. 60/520,844, filed Nov. 18, 2003 and 60/541,409, filed Feb. 3, 2004, each of which is herein incorporated by reference in its entirety.

### FIELD OF INDUSTRIAL APPLICABILITY

The invention relates to a method for continuous production of hydroxyazapirones, for example 6-hydroxybuspirone, using a modified reactor. The products of the invention are useful as pharmaceutical agents.

### BACKGROUND OF THE INVENTION

Certain azapirones, when hydroxylated, are well known to have therapeutic potential, particularly in treating anxiety disorders and depression. The hydroxylated azapirones of particular interest have the formula: 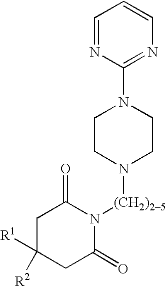
in which R¹ and R² are independently hydrogen or C₁₋₆ alkyl, or where R¹ and R² taken together form a ring, and n is an integer from 0 to 5.

A batch oxidation method for producing the hydroxylated compound is disclosed in commonly assigned PCT Patent Application No. WO 03/24934, filed on Aug. 7, 2003 by Jeffrey DePue, Atul Kotnis, Simon Leung, Eric Dowdy and Daniel Watson and entitled “Improved Process for Hydroxyazapirones”. That application, the contents of which are here incorporated by reference, discloses several prior art methods of producing the desired end product and reveals a new and improved process which involves the batch treatment of the azapirone compound with oxygen to produce the desired hydroxyazapirone compound via an imide enolate anion intermediate. That oxidation is necessarily carried out at an exceedingly low (cryogenic) temperature, typically between about −40° C. to about −100° C., preferably to a range of about −68° C. to about −75° C. Carrying out that batch process at higher temperatures results in the use of undesirably large amounts of solvents, and in excessive production of impurities, which are particularly difficult to remove. Accordingly, for best results the process requires that the oxidation reaction be conducted at a temperature below −60° C. Moreover, because the oxidation reaction could produce explosive peroxides, which cannot safely be present within the reaction vessel in large amounts, the size of each batch is required to be limited. These factors can cause a significant increase in production costs. In addition, maintaining cryogenic temperatures, for example on the order of about −70° C., is quite difficult and costly, and limitations on the size of a given batch means that either production-rate is limited or a plurality of costly pieces of equipment are required.

It is therefore an object of the present invention to provide a continuous process for the making of hydroxyazapirone compounds by carrying out the formation of an imide enolate anion followed by the oxidation of that anion, and optionally also other related steps in the overall production process, in a continuous fashion.

It is a further object of the present invention to provide a continuous process for the making of such compounds which can be carried out at a significantly higher (less cold) temperature than is required when the formation of the imide enolate and the oxidation are performed batchwise, while at the same time maintaining the desired degree of purity of the end product.

It is yet another object of the present invention to provide such a process in which productivity is increased while maintaining efficiency and satisfactory yield and purity of the final product.

It is a further object of the present invention to provide such a process in which, as a further savings, the volume of required reactants such as solvents is decreased.

It is therefore an overall object of the present invention to provide a method of making hydroxyazapirones by oxidizing imide enolate anions which is cheaper, faster and more productive than the previously known batch process.

To the accomplishment of the above, and to the accomplishment of such other objects as may hereinafter appear, this invention relates to a continuous process for the production of hydroxyazapirones by oxidation, as set forth herein.

### SUMMARY OF THE INVENTION

Generally, the invention comprises a continuous reaction process for the preparation of a hydroxyazapirone compound from an imide enolate anion by oxidation of said anion, which comprises carrying out said in a continuous reactor through which said imide enolate anion and oxygen, while being cooled, are contacted as they continuously flow along the length of said reactor. In certain preferred embodiments, the preparation of the imide enolate anion may additionally be conducted in a continuous reactor.

The enolate formation step, or enolization, is carried out in stoichiometrically controlled fashion, meaning herein that the proportions of the reactants, in particular the base reactant, is controlled to ensure optimal conversion of the azapirone to the enolate anion and thus the final product.

The invention further comprises a process of making a hydroxyazapirone compound from an imide enolate anion by oxidation of said anion which comprises (a) continuously producing said anion in a first reactor under stoichiometrically controlled conditions, (b) continuously feeding that anion through a second reactor where it is oxidized by contact with an oxidizing agent, (c) quenching the oxidized anion and (d) recovering the hydroxyazapirone compound

It has now been found that if the imide enolate anion and the oxygen are continuously brought together to react, which may readily be accomplished in a continuous reactor with counter current or co-current flow of the enolate and oxygen, not only is the desired hydroxyazapirone compound effectively produced but, the continuous oxidation can be carried out at much higher, i.e. less cryogenic temperatures than was required for the aforementioned batch process while still producing an end product of suitable purity. The continuous reactor used in this phase of the continuous process will desirably have a very small internal volume compared to the total volume of end product, and so large quantities of potentially unstable substances are not produced at any one time. Moreover, the use of a continuous reactor in either the enolate formation or oxidation phase of the process as described herein in conjunction with a cooling process provides a very high heat-transfer capability, thus reducing the possibility of the occurrence of a hot spot and adding to the thermal efficiency resulting from the continuous nature of the process.

Continuous reactors can be used in either the enolization or oxidation steps of the process. In certain embodiments of the invention, multiple reactors can be used in sequence to conduct the enolization and oxidation steps. Alternatively, the enolate preparation can be conducted by batch preparation, as is for example described in Application WO 03/24934, and the enolate then input to a continuous reactor for the oxidation step. Regarding the type of continuous reactor for the oxidation, any such reactor that allows contacting of a gas with a liquid while controlling the temperature of the reaction within the reactor chamber may be used. Non-limiting examples of such continuous reactors include falling film, trickle bed or bubbling reactors. Regarding the type of continuous reactor for enolization, any such reactor that allows contact between two liquids using static mixers or mechanical agitation followed by an extension of residence time to allow the completion of the reaction may be used.

Also, it has been discovered that the continuous process of this invention results in a significant reduction in the amount of solvent needed to dissolve the imide enolate anions before carrying out the oxidation step. In addition, because the oxidation in the continuous process takes place at a higher temperature than in a batch process, the reaction time is considerably reduced and faster reactions occur, thus further improving throughput.

The oxidation of the enolate is exothermic, and the heat produced tends to cause the reaction temperature to increase. An external cooling device, for example as in a jacketed reactor, may be used to maintain the desired low temperature of the reacting mixture. The desired cooling temperature according to the invention is, however, not in the range of the cryogenic temperatures used in the batch process. The term “cryogenic” as used herein means any temperature below −60° C. Observation of the temperature of the oxidation reaction at different points along the length of a trickle-bed reactor has disclosed that, under normal conditions, where a uniform cooling effect is exerted on the entire length of a reactor column of uniform size along its length, the temperature of the reagent stream increases significantly along its path as it moves through the initial phase of the reactor (as the oxidation begins), but decreases thereafter. However, this phenomenon can be greatly ameliorated by modifying the intensity of the cooling effect along the length of the chamber so as to apply a greater cooling effect at the top of the column near where the anion enters and a lesser cooling effect therebelow, This may be done by providing different temperature zones along the length of the column. Providing a lower temperature of the coolant in the initial phase maintains the temperature of the reaction within desired limits and comparatively uniform along the length of the column, increasing productivity and preventing excessive impurity production. It has been found that one way of accomplishing that result is by varying the geometry of the reactor column in accordance with the temperature profile of the reaction, as is disclosed in co-pending U.S. patent application Ser. No. 60/510,984, filed on Oct. 14, 2003 by Mourad Hamedi, Thomas L. LaPorte and Yeung Chan, entitled “Method and Apparatus for Optimizing Throughput in a Trickle-Bed Reactor”, the contents of which are herein incorporated by reference.

### BRIEF DESCRIPTION OF THE DRAWINGS

FIG. 1 is a graphical representation of the use of an inline infrared monitoring system to follow the progress of the oxidation of an azapirone compound (buspirone) in a continuous reactor.

FIG. 2 depicts the effect of changes in the base flow rate while that of the buspirone solution is maintained constant.

### DETAILED DESCRIPTION OF THE INVENTION

The formation of hydroxyazapirones according to various embodiments of the invention is described in Scheme 1. 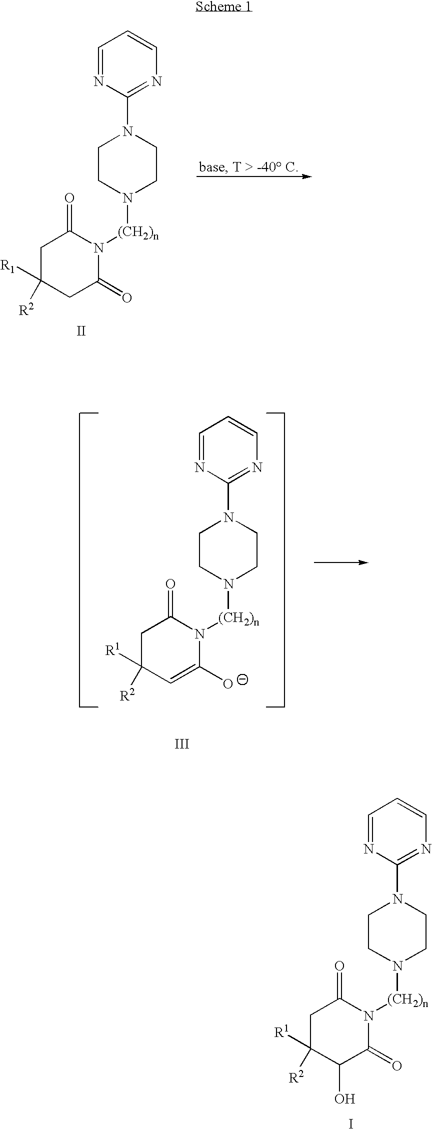
wherein R¹ and R² are independently hydrogen or C₁₋₆ alkyl, or where R¹ and R² taken together are —CH₂(CH₂)₀₋₅CH₂—, and n is an integer from 0 to 5, preferably 2 to 5. In certain preferred compounds made according to the process of the invention, R¹ and R² taken together form 1,4 butandiyl and n is 4 (6-hydroxybuspirone), or R¹ and R² are methyl and n is 4 (3-hydroxygepirone). The process of the invention provides an improvement in this synthesis in both the formation of the imide enolate anion (III) and in its conversion to the hydroxyazapirone product (I).

The azapirones that preferably serve as the starting materials in the process of this invention are defined by Formula I: 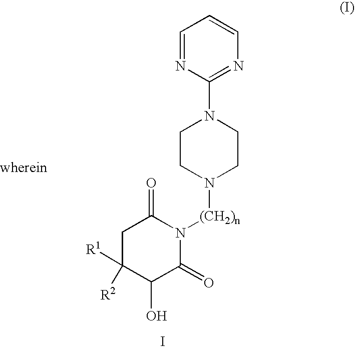
R¹ and R² are independently hydrogen or C₁₋₆alkyl, or where
R¹ and R² taken together are —CH₂(CH₂)₀₋₅CH₂—, and
n is an integer from 0 to 5. However, it should be recognized that a variety of reactions, in particular exothermic reactions, could be conducted using similar reactors and processes as generally described herein. The various preferred compounds of Formula I wherein n is from 2 to 5 can be hydroxylated to form 6-hydroxybuspirone: 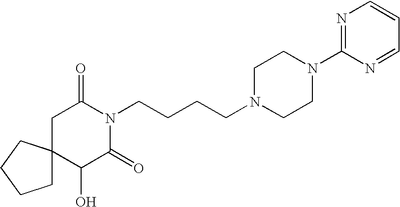
   wherein R¹ and R² of Formula I, taken together, form 1,4-butanediyl and n is 4; and 3-hydroxygepirone: 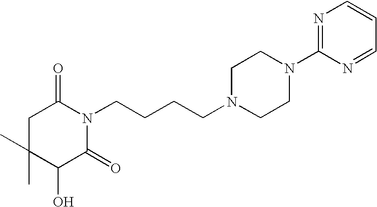
   where R¹ and R² of Formula I are each methyl and n is 4. The process will be herein exemplified in connection with the production of 6-hydroxybuspirone, but it should be appreciated that a comparable process can be carried out with respect to the production of 3-hydroxygepirone using gepirone as the starting material, as well as other compounds within the scope of Formula I. The preparation of 3-hydroxygepirone may be conducted according to the process of Scheme 2.

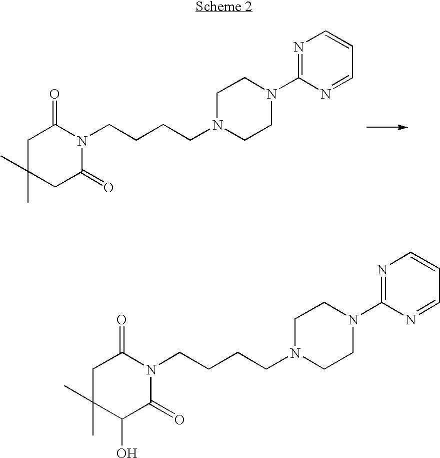

Commonly assigned and co-pending PCT Patent Application No. WO 03/24934 describes a batch process for the preparation of 6-OH buspirone using buspirone as the azapirone starting material. It should be noted that the materials and reagents recited in that disclosure, to the extent they are not itemized herein, should be interpreted as fully interchangeable with those here mentioned for the purpose of defining the range of equivalents within the scope of this invention.

The first step in the process is to produce the imide enolate anion that is subsequently to be oxidized. To that end, the azapirone is dissolved in an appropriate solvent containing preferably 1-5 equivalents of a suitable reductant to which a strong base is added. Many solvents are suitable for enolate generation, including the ethereal solvents tetrahydrofuran, diethylether, 1,2-dimethoxyethane, dioxane, and 2-methyltetrahydrofuran. Tetrahydrofuran (THF) is a preferred solvent for this reaction. A suitable reductant in the range of 1-5 equivalents may be added to the solution. Suitable reductants are those that reduce organic hydroperoxides to alcohols. Preferred reductants include tri-(C₁₋₉)alkylphosphites. Other reductants, such as triarylphosphites, triaryl- and trialkyl phosphines, thiourea, sodium borohydride, copper (II) chloride with iron (II) sulfate, iron (III) chloride, titanium isopropoxide, dimethyl sulfide, diethyldisulfide, sodium sulfite, sodium thiosulfate, zinc and acetic acid, and 1-propene, may also be used. While the reductant may be added at any convenient stage of the process, it is preferably present when the oxygenation reaction is initiated. About one equivalent of an appropriate strong base is then added. The base mediates deprotonation and formation of an imide enolate anion, which may be associated in situ with the base cation, M⁺, M being representative of any species that forms a cation upon reaction or dissociation of the base. Preferred bases suitable for this type of deprotonation include disilazanes, such as lithium bis(trimethylsilyl)amide, sodium bis(trimethylsilyl)amide, and potassium bis(trimethylsilyl)amide. Other strong bases may be used including dialkylamide bases (such as lithium diisopropylamide), metal hydrides, and metal alkoxides.

These reactions may be carried out at temperatures ranging from about ambient to about −80° C. When, as is preferred, this stage in the process is carried out continuously, the reagents flowing through the system can be either pre-cooled and then mixed, or mixed and cooled at the same time, or mixed at ambient temperature for a very short period of time (typically less than a minute) and then cooled. The mixing apparatus can be a jacketed or unjacketed tube equipped with a static mixer. For the case where cooling is applied, the jacket is held at a temperature of about −17° C., preferably. The mixing using a static mixer can be followed by a inline mechanical mixing if needed. After the reaction stream is fully mixed it passes through an extension of the residence time for enolization by further cooling in a multi-tube heat exchanger, so that the product leaving this stage is at a temperature between approximately −28° C. and −40° C. The product enters the oxidation reaction in this approximate temperature range.

The next step in the process is oxidation, which changes the anion to the desired end product, such as 6-hydroxybuspirone. In preferred embodiments, an in-situ reductant such as described previously is present in order to assure the success of the reaction. Without a reductant, large quantities of difficult to remove impurities may be formed. Oxidation takes place by causing oxygen to continuously react with the imide enolate anion while the anion travels from the inlet to the outlet of a suitable vessel. This is preferably accomplished, in one embodiment, by using a trickle-bed reactor, with the anion continuously flowing downwardly and with the oxygen continuously flowing upwardly or downwardly along the length of the reactor. For example, the oxidation can be carried out in a trickle-bed reactor consisting of a uniform column 60″ long and ⅞″ in internal diameter, surrounded by a cooling jacket through which refrigerated coolants are continuously circulated, preferably upwardly along the column. Such a reactor is illustrated in U.S. application Ser. No. 60/510,984. The coolant employed when it enters the jacket surrounding the column may be in the range of −34° C. to −39° C., while the oxygen flow through the column may be at a rate of approximately 800-1200 ml/min. Oxygen gases are the preferred sources of molecular oxygen, but other sources may be used, such as molecular oxygen in the form of gaseous mixtures. The reactor can be operated at ambient pressure or at higher pressure. A higher pressure will increase the solubility of oxygen in the liquid stream thus increasing the oxygen transfer rate (and the reaction rate).

Use of the continuous process of the invention advantageously allows the oxidation and enolization be carried out at a higher, i.e. less cryogenic, temperature than the batch process. The prior art batch process is carried out preferably at −70° C., a cryogenic temperature that is difficult and costly to the maintain, and each batch requires approximately 8 to 24 hours to complete oxidation, depending on scale. Attempts to perform the batch process at higher temperatures (under less cold conditions) resulted in the production of an impermissible amount of impurities which are difficult to remove. In addition, the oxidation process involves the production of intermediates such as hydroperoxides which are thermally unstable and which may produce serious explosions. Hence the batch process not only as a practical matter must be conducted at cryogenic temperatures, but even at those temperatures the reaction may be difficult to control.

In marked contrast, the continuous oxidation process of the present invention is carried out at much higher (less cold) temperatures while reducing the occurrence of impurities, and can be run under more thermodynamically controlled conditions. Typically, the enolate anion is formed at a temperature between ambient and −40° C. The temperature of the reaction stream containing the enolate anion at the inlet of the oxidation reactor is maintained at between −28° C. and −40° C. whereas the temperature inside the oxidation reactor can be allowed to go as high as −15° C. Preferably the temperature inside the oxidation reactor is maintained between −35° C. and −18° C. The temperature inside the enolization and oxidation reactors is a function of the reactor geometry, reactant stream flow-rates and coolant flow-rates. The coolant temperature is not the sole controlling parameter.

Moreover, productivity using the invention is not limited by vessel size or containment capacity. In the continuous reaction of the present invention, the surface area to volume ratio is maintained constant as the reactor is scaled up. In the batch process, productivity is limited by the size of the batch vessel, which if excessively large would present undesirable, proportionate exothermic generation of energy, as well as manipulation and production problems. The larger vessel would be more difficult to manipulate and would require a longer time for the oxidation of the contents to be complete. For example, in a lab-scale set-up using this continuous process, a flow-rate range of 88 to 125 ml/min could be achieved while generating product within purity specifications, leading to a continuous productivity of about 11.2 kg of the end product per day. An added advantage of the continuous process is that the amount of solvent, such as tetrahydrofuran, can be reduced, in this example from about 24.9 ml/g for the batch process to about 15.1 ml/g for the continuous process.

It has additionally been observed that generation of a stoichiometric amount of enolate is a control point for optimizing the instant process. Specifically, undergeneration of enolate results in poor conversion, higher amounts of recovered starting material and lower yield, while overaddition of base results in the production of dihydroxylated side products as impurities. Regarding the type of continuous reactor for the oxidation, any such reactor that allows contacting of the various reactants as required in the overall process would be suitable. For a given stoichiometry, the amount of impurities formed and the amount of remaining starting material are functions of the efficiency of the enolization and oxidation reactors, as well as of the work-up and extraction procedures that are subsequently performed after the oxidation step. Controlling residence time for a given operating temperature will control reactor efficiency.

In general, the base charge to the enolization reactor, relative to the amount of starting material, could be at a ratio in the range of 0.92-1.02. A preferable stoichiometry would reflect approximately 0.97-0.99 equivalents of base relative to starting material; however, as noted, the reaction will run successfully with the larger variations in the stoichiometry. For example, the presence of water in the starting material solution would require higher amounts of base.

Accordingly, the enolate formation is suitably monitored to ensure maximum yield is obtained while limiting the generation of unwanted side products. In this regard, various forms of reaction monitoring may be used. In particular, FTIR may be employed to directly observe conversion of the starting imide to the corresponding enolate. Direct observation of anion generation allows increase in the flow rate of the base until the IR signal associated with the starting material no longer declines, thus indicating complete consumption of the starting material. The flow rate of the base is then incrementally reduced until a signal for starting material is observed. Correlation of this signal with the purity profile using analytical HPLC provides a product stream with desirable purity/impurity characteristics. This desirable profile in turn indicates the desired flow rates for the starting material and base streams that should optimally be used in the process. In this manner, any deviations in the desired flow rates of the starting material and the base streams can be monitored and thus correlated to the optimal reagent stoichiometry.

In addition to the monitoring conducted in the oxidation step, reaction monitoring may also be used to monitor in-line or offline monitoring of the formation of product as the process proceeds, thus allowing for adjustments to the quantities of reagents and flow-rates as needed. In any such application, this monitoring may be accomplished by any means generally known in the art, including but not limited to spectral monitoring of molecular reactants or particulates, or by chemical analysis such as LC, HPLC, Raman, mass spectrometry. In certain embodiments of the invention, an infrared monitoring system (for example the REACT IR™ system developed by Mettler Toledo International Inc., United States), may be used in an in-line or off-line configuration to monitor the progress of the reaction, typically from the initial charge of the starting materials through generation of an optimal yield of product.

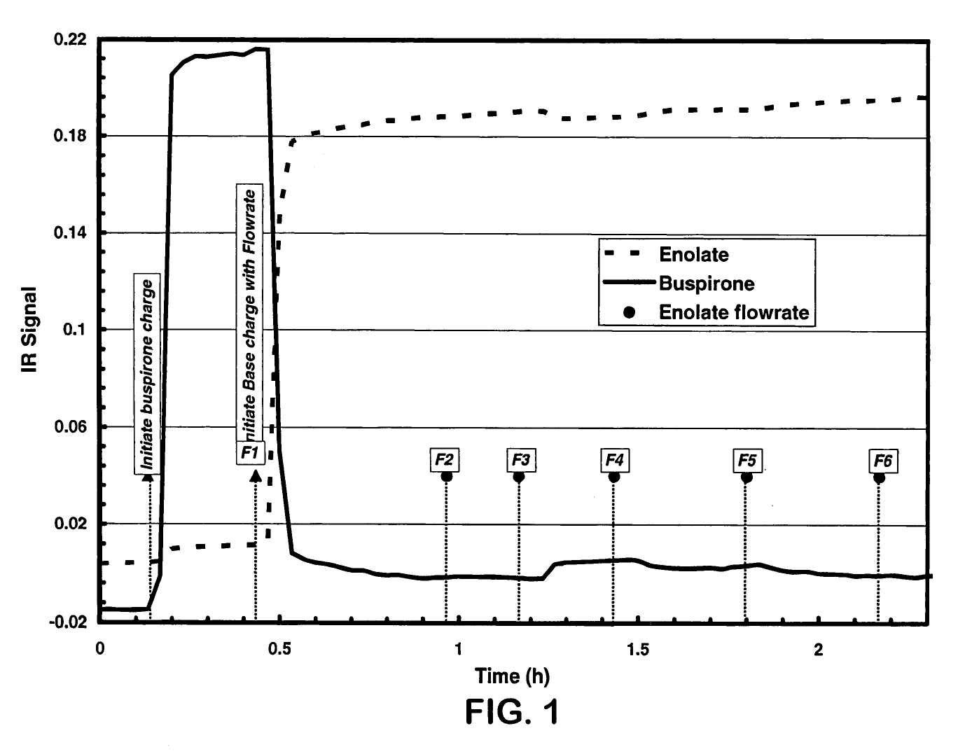

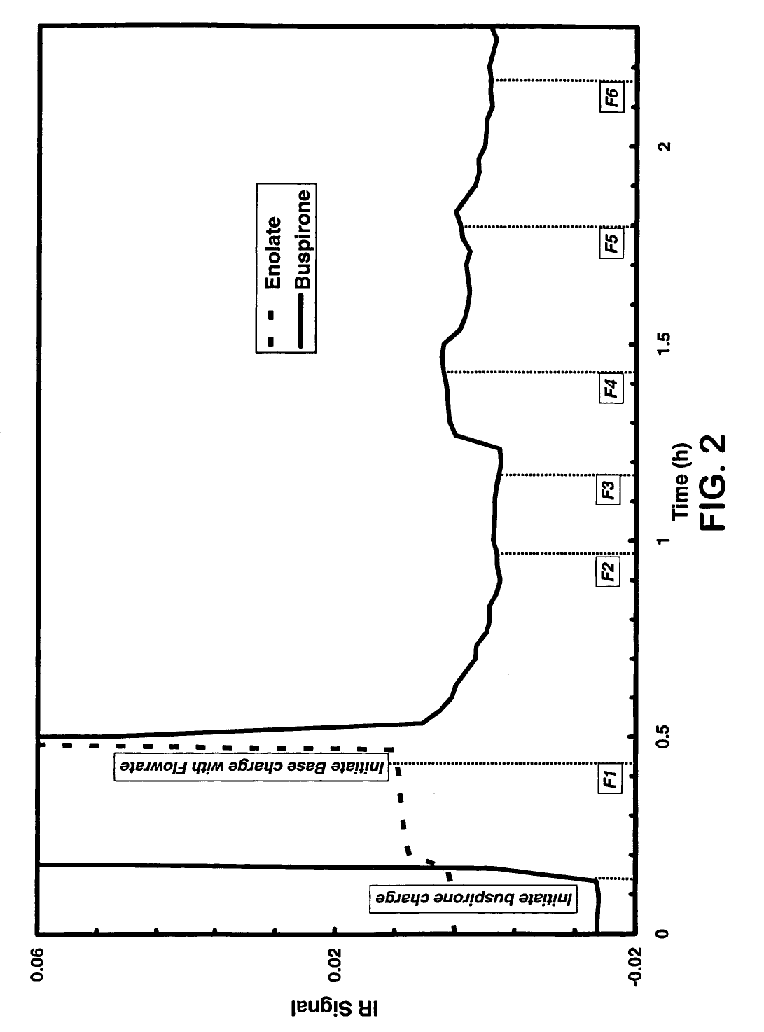

FIGS. 1 and 2 show the concentration profile of enolate and buspirone as a function of time. As the buspirone solution starts flowing through the system, its IR signal is shown to increase (solid line). When the base flow is initiated, the IR signal of buspirone drops while that of the enolate increases. FIG. 1 exemplifies the progress of a reaction from the point of initial charge of buspirone starting material through multiple charges of base reagent throughout the reactor, as determined by the changes in IR signal strength over time. In FIG. 1, the dotted lines indicate the time at which the flow rate of the base was modified.

While FIG. 1 shows a general view of the concentration profiles, IR can also be used, for example, to enable observation of deviations of the flow rates, thus allowing real time adjustments in flow rates to be made. FIG. 2 shows the effect of minute changes of the base flow rate while that of the buspirone solution is maintained constant. These diagrams exemplify how an IR monitoring system (e.g. the REACT IR™ technique can be used to easily detect 1% changes in the base flow rate (e.g. from flow rate F3 to Flow rate F4 in FIG. 2). Also the REACT IR™ technique can be used to detect overcharge of base since no change in enolate signal is detected as the flow rate is increased from F1 to F2 (FIG. 1).

The foregoing described methods when used in various combinations and applications thus result in an optimized process, which provides the product in high quality and yield. It is an observed advantage of the invention that the continuous nature of the present process enables off-line and on-line monitoring of the product as the process proceeds, thus allowing for adjustments of quantities of reagents and flow-rates as needed.

After oxidation the resultant product is then quenched by being diluted with a suitable solvent such as methyl tert-butylether, ethyl acetate, or 2-methyl-tetrahydroxyfuran, warmed to room temperature and neutralized, for example, with 1 M hydrochloric acid until the pH is about 6.0 to 7.0, preferably about 6.5 to 6.9. Other acids may be used, and the pH may also be adjusted with various bases such as sodium phosphate. This may be accomplished by feeding the oxidized output, to which nitrogen may be added, into quenching vessels, there to be mixed with a solvent and acid and allowed to stand. In essence, the quenching can therefore also be performed in a continuous fashion.

An alternative workup and isolation protocol may be followed after the completion of oxidation. In this procedure, the reaction mixture is treated with acid to lower the pH to approximately 2.0, whereupon the mixture is heated to hydrolyze the residual triethylphosphite to phosphorous acid and monoethyl phosphorous acid. Neutralization with base followed by aqueous extraction removes the phosphorous acids. Solvent exchange from tetrahydrofuran into isopropanol at reduced pressure followed by crystallization affords the desired 6-hydroxybuspirone in yields of about 70% with very good purity (typically around 97% purity).

Detailed observation of the temperature of the reaction mixture as it proceeds down the mixing column of a trickle-bed reactor indicates that, presumably because of the exothermic nature of the oxidation reaction, there is an initial temperature increase reaching a maximum centerline temperature of −16° C. in the first foot of travel through the reactor, with the temperature of the stream decreasing to around −35° C. in the remaining length of the reactor. This reduction is probably due in part to the fact that the concentration of the enolate decreases as oxidation proceeds.

An optimized reactor suitable for use in the process of this invention would be one in which the temperature is maintained within a relatively narrow temperature range along the entire reactor length. This can be accomplished by providing a plurality of cooling sources along the length of the column, with the earlier source providing a greater cooling effect than the later source. For example, the coolant applied to the first half of the reactor length may be at −37° C. while the coolant applied to the remainder of the length of the reactor may be at a somewhat higher temperature such as −32° C. to −34° C. Another and preferred approach is that disclosed in the aforementioned Hamedi, et al., patent application Ser. No.60/510,984, where the diameter of the first half of the column length is less than the diameter of the second half.

The continuous oxidation production process here disclosed, when compared with the prior batch process, resulted in a significant increase in productivity and no appreciable increase in impurity production. The operating temperatures used for the oxidation reaction were significantly higher than those required for the batch process, resulting in significant savings in equipment and operating costs. In addition, a significant reduction in solvent usage also was achieved. The use of higher temperatures in the continuous process of the present invention produces faster reactions, and therefore increased productivity in comparison to the batch process. Because at any given moment during production there is a smaller amount of material in reaction, there is less risk of productivity loss due to equipment failure.

6-hydroxybuspirone produced according to the invention is useful as an anxiolytic or antidepressant agent in the treatment of patients with anxiety and depression disorders, as is disclosed in commonly assigned PCT Patent Application WO 01/52853, which is herein incorporated by reference. The compound may additionally be used in combination with other therapeutics, for example for treatment of pain as is disclosed in commonly assigned U.S. Pat. No. 6,566,361, herein incorporated by reference.

### EXAMPLES

### Example 1

### Continuous Oxidation of Buspirone

A continuous oxidation to produce 6-hydroxybuspirone was carried out as follows: to produce the imide enolate anion, a mixture of buspirone free base, triethyl phosphite, and tetrahydrofuran (THF), at a rate of from 73-103 mL/min was mixed with NaHMDS (sodium hexamethyldisilazide) in THF flowing at a rate of 15-21 mL/min of NaHMDS and THF. These reagents were initially combined at approximately 20° C. and, while flowing, were continuously mixed and cooled to about −35° C. Cooling was accomplished in a static mix column followed by a multi-tube heat exchanger. The temperature profile along the length of the column was controlled by utilizing a trickle-bed reactor the first half of the length of which had an internal diameter of ⅞″ and the second half of which add an internal diameter of 1⅞″, and the total length of which was approximately 70″, with a single coolant flow from bottom to top, as described in patent application Ser. No. 60/510,984. For a reactor of approximately the same length, the result was a 3-fold increase in throughput and a slight improvement with regard to the impurity profile of the stream when compared with the output of a column of similar length and uniform diameter with a single coolant flow.

### Example 2

### Continuous Oxidation of Buspirone Employing In-Situ Infrared Monitoring

A solution of buspirone, THF (15 mL/g) and triethyl phosphite (3.5 eq.) was passed through a static mixer (32-37 mL/min). In-line React-IR™ monitoring was implemented to observe the starting material signal. The THF solution of NaHMDS (1.0 M, 15-21 mL/min) was then started while maintaining the temperature of mixing in the static mixer at approximately −33° C. to −38° C. Small increases in the flow rate of the sodium bis(trimethylsilyl)amide were then performed until the IR signal for buspirone reached a minimum indicating complete deprotonation of buspirone generating the enolate of buspirone. The flow rate of the sodium bis(trimethylsilyl)amide solution was incrementally reduced until the buspirone IR signal indicated a 0.5% to 5% excess of buspirone (preferred range is 1-3% excess buspirone). Correlation of this signal with the purity profile using analytical HPLC provided a product stream with desirable purity/impurity characteristics.

This continuous flow enolate solution was then passed down (40-45 mL/min) a 60″×⅞″ jacketed stainless steel column packed with Pro-Pak stainless steel packing material while oxygen gas (0.4-1.0 L/min) was introduced at the opposite end of the column in a counter-current fashion. The jacketed stainless steel column was kept at a temperature between −28 to −40° C. through the use of a heat exchanger. The solution emerging from the column was quenched into a mixture of 1 M HCl and MTBE.

Work-up and isolation were done according to the methods described in PCT Patent Application No. WO 03/24934, previously herein incorporated by reference.

While only a limited number of embodiments of the present invention have been specifically disclosed herein, it will be apparent that many variations may be made therein, all without departing from the spirit of the invention as defined in the following claims.

1. A continuous reaction process for the preparation of a hydroxyazapirone compound from an imide enolate anion by oxidation of said anion, which comprises carrying out said oxidation in a continuous reactor through which said imide enolate anion and oxygen, while being cooled, are contacted as they continuously flow through the reactor.

2. The process of claim 1 wherein the reaction comprises preparation of compounds of Formula I 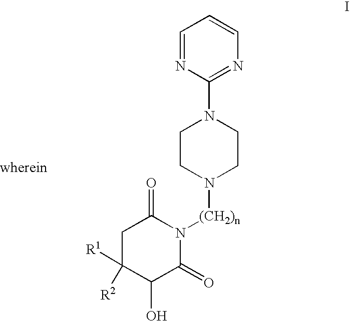
R¹ and R are independently hydrogen or C₁₋₆alkyl, or where
R¹ and R² taken together are —CH₂(CH₂)₀₋₅CH₂—, and
n is an integer from 0 to 5;
comprising reacting a compound of Formula II 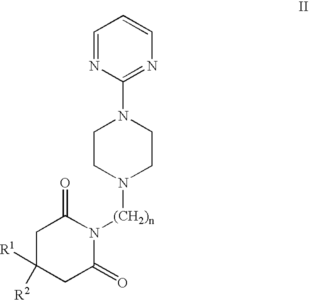
with a strong base under stoichiometrically controlled conditions to form an anion according to Formula III 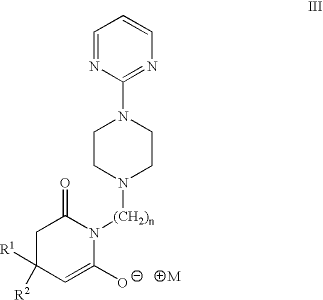
where M⁺ is the base cation; and oxidizing the anion of Formula III to produce a compound according to Formula I.

3. The process of claim 1, further comprising preparation of the imide enolate anion in a continuous reactor under stoichiometrically controlled conditions.

4. The process of claim 2, in which n is an integer from 2 to 5.

5. The process of claim 1, in which the temperature of the imide enolate anion stream at the inlet of the oxidation reactor is between about −28° C. and about −40° C.

6. The process of claim 1, in which the temperature at which the imide enolate anion is formed is between ambient temperature and approximately −40° C.

7. The process of claim 1, in which the temperature at which the imide enolate anion is oxidized in the reactor is between approximately −15° C. and −40° C.

8. The improvement in the process of making a hydroxyazapirone compound from an imide enolate anion by oxidation of said anion which comprises (a) continuously producing said anion in a first reactor under stoichiometrically controlled conditions, (b) continuously feeding that anion through a second reactor where it is oxidized by contact with an oxidizing agent, (c) quenching the oxidized anion and (d) recovering the hydroxyazapirone compound.

9. The process of claim 8, in which said formation of the imide enolate anion is carried out at a temperature of from ambient temperature to about −40° C.

10. The process of claim 8, in which the temperature at which the imide enolate anion is formed is between approximately −40° C. and −15° C.

11. The process of claim 8, in which the temperature at which the imide enolate anion is oxidized in the second reactor is between approximately −28° C. and −40° C.

12. A process for making a hydroxyazapirone compound from an imide enolate anion by oxidation of said anion which comprises (a) continuously producing the imide enolate anion under stoichiometrically controlled conditions, (b) continuously feeding the imide enolate anion through a second reactor where it is oxidized by contact with an oxidizing agent, (c) quenching the oxidized anion and (d) recovering the hydoxyazapirone compound.

13. The process of claim 12, in which said formation of the imide enolate anion is carried out at a temperature of from ambient temperature to about −40° C.

14. The process of claim 12, in which the temperature at which the imide enolate anion is formed is between approximately −40° C. and −15° C.

15. The process of claim 12, in which the temperature at which the imide enolate anion is oxidized in the second reactor is between approximately −28° C. and −40° C.

16. The process of claim 2 wherein the compound of formula I is 6-OH buspirone

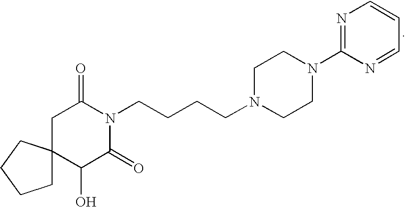

17. The process of claim 2, wherein the compound of Formula I is 3-hydroxygepirone

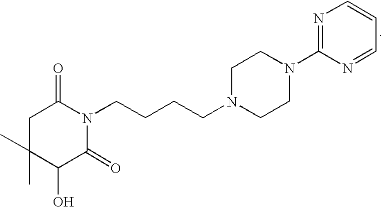

18. The process of claim 2, wherein the conversion of the compound of Formula II to the anion of Formula III is monitored using IR spectroscopy.

19. The process of claim 18 wherein the conversion of the compound of Formula II to the anion of Formula III can be correlated with the HPLC purity/impurity profile of the product stream containing the compound of Formula I.
